(19) United States
(12) Patent Application Publication
(10) Pub. No.: US 2026/0245710 A1
(43) Pub. Date: Aug. 20, 2026

(54) METHOD FOR GENERATING AND OUTPUTTING A WORK INSTRUCTION AND MEDICAL SYSTEM

(71) Applicant: B. Braun Melsungen AG, Melsungen (DE)

(72) Inventor: Markus Kullmann, Alheim (DE)

(21) Appl. No.: 19/537,290

(22) Filed: Feb. 11, 2026

(30) Foreign Application Priority Data
Feb. 17, 2025 (EP) 25158374.6

(51) Int. Cl. G16H 40/40; A61B 90/96; A61B 90/98; G16H 40/20; A61B 90/00

(52) U.S. Cl. CPC G16H 40/40; A61B 90/96; A61B 90/98; G16H 40/20; A61B 2090/0809

(57) ABSTRACT

A method for generating and outputting a work instruction includes the steps of recording device-specific information from a medical device and/or a component of the medical device, reading out or retrieving repair-relevant information based on the recorded device-specific information, in particular from a central data processing unit, deriving the work instruction from the read out repair-relevant information, outputting the derived work instruction to a user, in particular via an output unit, and checking, in particular by the medical device, whether the task has been completed.

### CROSS-REFERENCE TO RELATED APPLICATION

This application claims priority under 35 U.S.C. § 119 to European Application No. 25158374.6, filed on February 17, 2025, the content of which is incorporated by reference herein in its entirety.

### FIELD

The present disclosure relates to a method for generating and outputting a work instruction and a system comprising a medical device and a central data processing unit.

### BACKGROUND

When servicing or repairing medical devices such as medical pumps or dialysis machines, service technicians follow a manual. During their product life cycle, medical devices undergo changes such as design modifications, component changes, or changes and updates to the software or operating system. These changes may also affect the repair or servicing of the devices and must therefore be reflected in the manual/service booklet. For example, two different components may only be compatible with each other, i.e., in combination. This means that if one of these components is replaced, the other component must also be replaced. Similarly, certain newly installed components may require a specific software version in order to function. The changes in the product life cycle thus create dependencies between different components, component variants, and/or software variants. These dependencies have an impact on the maintenance and repair of the devices. At present, these dependencies are mostly described in the manual depending on the serial number of the respective device or component. As the number of products and variants increases, the clarity of the manuals decreases significantly. It can be difficult and time-consuming for service technicians to find the correct repair or maintenance instructions for the right model or the right components. This is a source of error, takes more time, and thus leads to higher personnel and repair costs.

If new components are installed in a device, these new components can create new dependencies for the next repair or maintenance. For this reason, the product numbers of these new components are usually documented manually in a device logbook, which may be a device-specific manual or service booklet, for example. This is also a potential source of error.

Furthermore, there are sometimes strict documentation requirements in the field of medical technology with regard to the maintenance and repair of medical devices. Currently, maintenance or repair work carried out must be documented manually by service technicians or users. If this documentation is forgotten or carried out incorrectly, this may be a source of errors during further work on the device.

### SUMMARY

The purpose of the present disclosure is therefore to overcome or at least mitigate the disadvantages of the prior art and, in particular, to provide an (automated) method for providing repair instructions that are correct, tailored to the product in question, and as easy to follow as possible. Preferably, (automated) documentation of maintenance performed should also be provided.

The present disclosure relates to a method for (automatically) generating (device- or variant-specific) work instructions/repair guidelines/service guidelines/maintenance guidelines with the following steps: Device-specific information about a medical device and/or individual components/assemblies is recorded. Repair-relevant information based on the recorded device-specific information is read out/retrieved. The repair-relevant information can be read out/retrieved from a central data processing unit. Furthermore, the work instruction is derived from the repair-relevant information that has been read out/retrieved. The derived work instruction is then output to a user. It is preferable to check whether the task (or “measure”) has been completed or (correctly) implemented. The task of the present disclosure is further solved by a system comprising the medical device and the central data processing unit. The medical device displays device-specific information. The central data processing unit contains repair-relevant information about the medical device, which is preferably linked to device-specific information, for example in a database. The system disclosed is set up, for example via a control unit of the medical device and/or the central data processing unit, to perform the method disclosed.

In other words, the method disclosed generates a device-specific and/or variant-specific (step-by-step) plan for the repair/restoration/servicing/maintenance of a medical device. For this purpose, information is recorded that is suitable for assigning a (product) variant/version/model to the device. Based on the information displayed for the variant, further information is retrieved, which indicates how the corresponding variant is to be repaired or maintained. Based on this additional information about the repair or maintenance of the specific variant of the device, a (variant-specific) work instruction is derived/read out for exactly this variant of the device or the device assembly. The work instruction is output to the user or displayed to the user as a set of instructions.

Preferably, the work instruction can be derived from a control unit of the medical device and/or from a central data processing unit. However, the work instruction can also be derived from a separate device, such as a handheld device like a smartphone. In this case, the derived work instruction can be output via the handheld device screen. Alternatively, the work instruction can be output on the screen of the medical device.

The work instruction may, for example, be an instruction/guideline that tells the user how to perform a measure (on the medical device). The measure may involve, in particular, repair/servicing or maintenance/upkeep of the medical device. Maintenance/servicing may also include installing a software update or installing/assembling an extension to the medical device. By following the derived work instructions, error-free, quick, and smooth repair or maintenance can be ensured. Furthermore, following the work instructions ensures that the results of the measure are reproducible.

Device-specific information may include, in particular, information that allows the medical device to be clearly identified. The device-specific information allows a specific device to be assigned to a (product) variant/a model/a (software) version/a production line. Examples of device- specific information include, in particular, a serial number, a model number, and/or a unique identifier (UID). Furthermore, device-specific information may also include information that provides details about the installed software version or a software update, or previous repairs and/or maintenance. The device-specific information can either be stored on the medical device and retrieved from the medical device, or it can be entered by the user into an input unit (of the medical device).

The repair-relevant information may include, in particular, information indicating how a particular variant can be repaired or maintained. The repair-relevant information can specify, in particular, what has changed in the specific variant compared to another variant and how this change affects the repair/maintenance. In particular, repair-relevant information can specify which dependencies or links exist between individual (new) components/assemblies.

Specifically, such a link can indicate that a particular component is only compatible with another component from a certain product number onwards. This means that if one component is installed, it may also be necessary to replace the other component. This dependency must be derived manually from the manual using known methods. The repair-relevant information can store the dependency between the two components in a computer-readable form. The dependency may also be a link between a newly installed component and a software update that is necessary for the new component to function. In a specific example, a new screen for an infusion pump may only be compatible with a new version of an upper housing shell that has a keyboard. The relationship between the compatibility (product number) of the screen and the required version of the upper housing shell can be stored and retrieved as repair-relevant information. In this case, the derived work instruction may then include that the upper housing shell should also be replaced when the screen is replaced.

The medical device may in particular be a medical pump, such as a syringe pump or an infusion pump. The medical device may also be a dialysis machine or dialysis pump. Other embodiments of the medical device are also conceivable in principle.

The central data processing unit can be a server or a computer, in particular. Furthermore, the central data processing unit can also be a database or data storage device on which the device-specific information is linked to the repair-relevant information. By linking device-specific information with repair-relevant information, the information relevant to the repair of a specific device can be retrieved for that device from the central data processing unit.

The check as to whether the measure has been completed may in particular be carried out by the medical device (automatically), for example by suitable recording and/or scanning of whether the measure has been properly carried out and completed by a user. For example, suitable detection sensors and/or suitable scanning devices may be installed in the medical device. Furthermore, the user can confirm on the input unit that the measure has been carried out. To do this, the user can check a box on the input unit or press a confirmation button. The user can also confirm that the measure has been carried out (successfully) by taking photos and/or uploading a maintenance log.

The method can be designed so that step-by-step work/repair/servicing/maintenance instructions are output or displayed to the user on the screen of the medical device or handheld device. This has the advantage that the user is guided step-by-step through the work/repair/servicing/maintenance process and, ideally, is not overwhelmed with unnecessary information at any point.

Furthermore, it may be provided that the work/repair/servicing/maintenance instructions are displayed or illustrated to the user on the screen of the medical device or handheld device by means of moving or still images, such as photos or videos. This greatly simplifies the work/repair/servicing/maintenance process, as the next process step to be performed is displayed on the screen immediately before it needs to be carried out. The user is thus guided step-by-step through the work/repair/servicing/maintenance process in a convenient manner.

The method is also advantageously designed such that the work/repair/servicing/maintenance process, in particular the steps performed, are automatically documented, preferably by the central data processing unit or by the control unit of the medical device. For example, after each step/measure has been carried out, the central data processing unit or the control unit of the medical device automatically documents the step/measure. This minimizes or eliminates the risk of incorrect manual documentation.

The method preferably provides for the connection of additional systems via interfaces. For example, an enterprise resource planning or enterprise information and process system and/or a digital device log and/or a service portal can be connected to the central data processing unit and/or the medical device via interfaces. For example, data about the original construction status of the medical device or a component or product of the medical device can be provided by the enterprise resource planning or enterprise information and process system. This data may include, for example, an article, a manufacturer’s report, batch information, etc. In addition, data about any repairs to the medical device or a component or product of the medical device following delivery can be provided via the digital device logbook. Furthermore, data relating to instructions and configurations can be provided from a service manual, which is made available, for example, via a service portal. The method preferably provides, from the large amount of available data, in particular using databases, algorithms, AI, etc., clear and specific step-by-step (repair/work/servicing/maintenance) instructions that are valid for the product/medical device in question, i.e., it derives these instructions and displays them to the user on a screen.

In summary, the core of the disclosure lies in the fact that the information relevant for repairing a specific medical device can be read out and used to generate (individualized) repair instructions/work instructions for that specific medical device, with a check being performed to verify that the corresponding repair/maintenance/servicing/work measure has been completed.

The method described in the disclosure allows the individualized work instruction to be displayed quickly and easily (automatically). This allows the user, for example a service technician, to view the work instructions for the measure, in particular repair and/or maintenance, without having to manually read out the link between the current version of the medical device and its specific work instructions in the manual. This ensures that the user always has the correct work instructions for the variant to be repaired. This minimizes a potential source of error during the measure and also saves the user working time. Maintenance costs can be minimized thanks to the time saved. The successful implementation of the measure can be recorded, for example, by the medical device.

The method disclosed can be carried out in particular when the medical device is not connected to a patient. This means that before the measure (repair/maintenance) is carried out on the device, the connection to the patient is disconnected. The method disclosed is therefore not a method for the surgical or therapeutic treatment of the human or animal body, nor is it a diagnostic method performed on the human or animal body, since it is performed when no patient is connected to the medical device or is being treated or being given therapy by the medical device.

Preferably, the medical device can have an input and/or output unit and a control unit and be connected to the central data processing unit via a data connection. The data connection can be used in particular to transmit device-specific information to the central data processing unit. Specifically, this means that the medical device can transmit device-specific information or device-specific information about components of the medical device (directly) to the central data processing unit via the data connection, preferably either automatically when the medical device itself considers a repair/maintenance/servicing measure to be necessary or determines that it is necessary, or upon request/specification by the user. Based on the device-specific information transmitted, the repair-relevant information for the respective device can be read out from the central data processing unit. In particular, the repair-relevant information read out can be further processed by the central data processing unit and the derived work instruction can be transmitted to the medical device via the data connection (from the central data processing unit). The input and/or output unit can be used to enter or record device-specific information and to output work instructions, preferably step-by-step work instructions, to the user.

The device-specific information can either be stored in the medical device or entered via the input and/or output unit (by the user). The work instruction can also be derived from the repair-relevant information in the central data processing unit by the medical device.

For data transmission between the medical device and the central data processing unit, the medical device can be connected to an IT infrastructure and, in particular, can be connected to the central data processing unit via WLAN or LAN or a similar data connection for data transmission.

Preferably, the system can also have a handheld device. The handheld device may preferably be a smartphone or tablet computer or a similar handheld device with an input and an output unit. The handheld device can, in particular, record device-specific information from the medical device (via an input unit). For this purpose, the handheld device can optically read out device-specific information, in particular as a barcode. The handheld device can also convert a photo of a device-specific series of numbers into device-specific information. Alternatively, the handheld device can read out electromagnetic radiation from the medical device, for example in the form of an RFID tag/RFID chip, in order to record device-specific information.

The handheld device can transmit the recorded device-specific information to the central data processing unit, for example via LAN, WLAN, Bluetooth, or similar (wired or wireless) data transmission. The handheld device can output work instructions to the user, in particular via a screen.

The disclosed system can provide the user (automatically) with correct work instructions that are appropriate for the device to be repaired.

According to an optional aspect of the present disclosure, the device-specific information may be, for example, a serial number, a barcode, and/or an RFID tag. Preferably, the device-specific information can be recorded visually and/or electronically. The device-specific information can be stored electronically on the medical device and thus retrieved electronically. The device-specific information may also be located elsewhere on the medical device so that it can be read out by the user manually or using the handheld device.

Preferably, the step of recording the device-specific information may include scanning a barcode (on the medical device) with the handheld device and/or electronically reading out an RFID tag. By recording device-specific information, a specific product variant can be assigned to the medical device. The device-specific information can thus represent the link to the work instruction for precisely this variant/model.

According to a further optional aspect of the present disclosure, the repair-relevant information may be the information required for the repair and/or maintenance of a specific product variant/model. In particular, repair-relevant information can define the dependencies between the individual components/assemblies of the medical device. The repair-relevant information allows device-specific information to be linked to information on exactly how the device variant in question can be repaired or maintained.

Advantageously, the step of outputting the derived work instruction can include outputting the individual (repair) steps of the measure step by step. This allows the user to be guided step by step through the measure. This minimizes errors in the implementation of the measure.

Conveniently, after the step of outputting the derived work instruction, a result of the measure can be entered (by the user). The input can be made in particular by taking a photo of the result and/or by manual input/confirmation on the medical device or handheld device. This allows the success of the measure to be documented. A system prompt after the measure has been completed can also prevent the documentation from being forgotten or skipped.

According to a further optional aspect of the present disclosure, a result of the respective step of the measure can be entered after individual steps of the work instruction. This also allows the individual steps of the measure to be documented. A control system can thus evaluate whether the measure has been carried out correctly.

Preferably, the result can be transmitted to the central data processing unit and stored in the central data processing unit. This allows measures that have been carried out and the success of these measures to be stored centrally and retrieved at any time. The central storage of information makes it easy to verify and monitor measures that have been carried out.

The central data processing unit can preferably be a database that links the device-specific information with the repair-relevant information and thus with the work instruction. However, the work instruction can also be generated by applying algorithms and/or AI. This enables work instructions to be generated easily and automatically.

The task of the present disclosure is further solved by a computer-readable storage medium comprising commands which, when executed by a computer, cause the computer to perform the steps of the method according to one of the above aspects.

Furthermore, the present disclosure relates to a computer program comprising commands that, when executed by a computer, cause the computer to perform the steps of the method according to any of the above aspects.

### BRIEF DESCRIPTION OF THE DRAWINGS

FIG. 1 shows a schematic representation of a system according to an embodiment of the present disclosure;

FIG. 2 shows a flow chart of a method according to the present disclosure; and

FIG. 3 shows a schematic representation of a system according to a further embodiment of the present disclosure.

### DETAILED DESCRIPTION

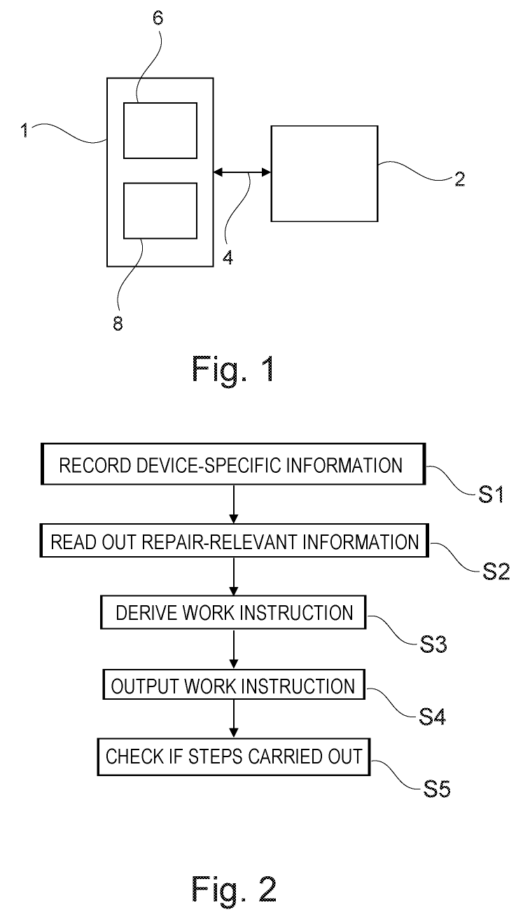

FIG. 1 shows a system with a medical device 1 and a central data processing unit 2. The medical device 1 is connected to the central data processing unit 2 via a data connection 4. The medical device 1 contains device-specific information. The device-specific information is transmitted via data connection 4 to the central data processing unit 2. Based on the device-specific information, repair-relevant information is read out from the central data processing unit 2. The read out repair-relevant information is evaluated either by a control unit 6 of the medical device 1 or by the data processing unit 2. Based on the repair-relevant information that has been read out, a (device-specific or variant-specific) work instruction is derived. The derived work instruction is output to the user via an output unit 8 of the medical device 1. Preference is given to step-by-step work instructions being output to the user via output unit 8 of medical device 1, preferably via moving images/videos.

FIG. 2 shows a flow chart of a method. In step S1, the device-specific information of the medical device 1 is recorded and (automatically or manually/upon request or prompt by a user) transmitted from the medical device 1 to the central data processing unit 2. Based on the device-specific information transmitted, repair-relevant information is read out from the central data processing unit 2 in step S2. In step S3, a work instruction is derived based on the repair-relevant information that has been read out. The derived work instruction is adapted/aligned to the transmitted device-specific information. The work instruction is therefore specific to medical device 1. In step S4, the derived work instruction is output to a user. The work instruction can in particular be a step-by-step instruction for the repair and/or maintenance of the medical device 1. In step S5, a check is performed to determine whether the measure/individual steps have been carried out (successfully). This check can be performed as a self-test by medical device 1. However, the check can also be performed by the user. The user can manually confirm successful completion. The method allows the (device-specific or variant-specific) work instruction to be generated quickly, easily, and automatically and output to the user.

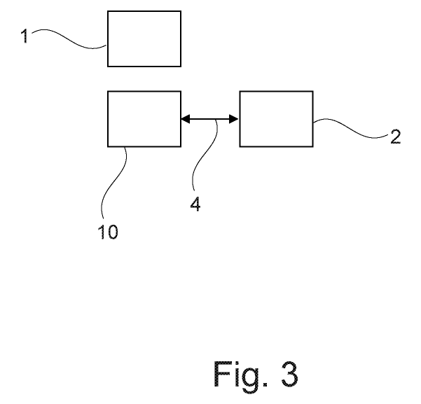

FIG. 3 shows a schematic representation of a system according to a further embodiment. The system comprises the medical device 1 and central data processing unit 2. The system also includes a handheld device 10, such as a smartphone or tablet. The handheld device 10 records device-specific information from the medical device 1 and transmits the recorded device-specific information to the central data processing unit 2. The repair-relevant information is evaluated either by the handheld device 10 or by the data processing unit 2. Based on the repair-relevant information that has been read out, a (device-specific or variant-specific) work instruction is derived. The derived work instruction is output to the user on a screen of the handheld device 10.

1. A method for generating and outputting a work instruction, the method comprising the steps of:
recording device-specific information from a medical device and/or a component of the medical device;
reading out or retrieving repair-relevant information based on the device-specific information;
deriving work instructions from the repair-relevant information that has been read out or retrieved; and
outputting the work instruction to a user.

2. The method according to claim 1, wherein the work instruction comprises an instruction for performing a task on the medical device.

3. The method according to claim 2, wherein the task comprises one or more of:
repair and servicing of the medical device, and
maintenance and upkeep of the medical device.

4. The method according to claim 2, wherein the step of reading out or retrieving repair-relevant information comprises retrieving repair-relevant information from a central data processing unit.

5. The method according to claim 4, wherein a result of the task is transmitted to the central data processing unit and stored in the central data processing unit.

6. The method according to claim 2, further comprising the step of entering a result of the task after the step of outputting the work instruction to the user.

7. The method according to claim 2, further comprising the step of checking whether the task has been completed.

8. The method according to claim 7, wherein the step of checking whether the task has been completed is performed using the medical device.

9. The method according to claim 2, wherein the step of outputting the work instruction includes stepwise outputting.

10. The method according to claim 9, wherein:
the work instruction comprises one or more work instruction steps,
the task comprises one or more task steps, and
a result of each task step is entered after completing each work instruction step.

11. The method according to claim 1, wherein the device-specific information comprises at least one of:
a serial number,
a barcode, or
an RFID tag.

12. The method according to claim 11, wherein the device-specific information is stored electronically in the medical device.

13. The method according to claim 1, wherein the step of outputting the work instruction to a user comprises outputting the work instruction via an output unit.

14. The method according to claim 1, wherein the step of recording device-specific information comprises optical or electronic recording.

15. The method according to claim 14, wherein the optical or electronic recording comprises scanning a barcode with a handheld device.

16. The method according to claim 14, wherein the optical or electronic recording comprises electronic reading out of an RFID tag with a handheld device.

17. A system configured to perform the method according to claim 1, the system comprising:
the medical device containing the device-specific information; and
a central data processing unit containing the repair-relevant information.

18. The system according to claim 17, wherein the medical device comprises device-specific information, an input and/or output unit, a control unit, and a data connection, the medical device being connected via the data connection to the central data processing unit, the medical device being configured to transmit the device-specific information via the data connection to the central data processing unit and outputs the work instruction via the input and/or output unit.

19. The system according to claim 18, further comprising a handheld device, configured to record the device-specific information of the medical device, transmit the device-specific information to the central data processing unit, and output work instructions to a user.

20. A computer-readable storage medium comprising commands which, when executed by a computer, cause the computer to perform the method according to claim 1.
